(19) United States
(12) Patent Application Publication
(10) Pub. No.: US 2007/0203516 A1
(43) Pub. Date: Aug. 30, 2007

(54) Apparatus and Methods for Creating an Opening in a Tissue Membrane

(75) Inventor: Asha Nayak, Menlo Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR (A Delaware Corporation), Santa Rosa, CA (US)

(21) Appl. No.: 11/276,323

(22) Filed: Feb. 24, 2006

(51) Int. Cl. A61B 17/34

(52) U.S. Cl. 606/185

(57) ABSTRACT

The current invention discloses an apparatus for creating openings in tissue membranes and methods for using the apparatus. The apparatus comprises a cutting device that can include a cutting element and a resisting element located on the ends of elongate members. The cutting device can be manipulated so that the cutting element and resisting element are on opposite sides of the target tissue membrane. The cutting device is delivered to a tissue membrane using an elongate delivery device that can be routed through a working lumen in an endoscopic probe. The delivery device can also access the tissue membrane via means other than an endoscopic probe. Embodiments of the invention can include a vacuum lumen in the delivery device and a vacuum source for stabilizing the tissue membrane relative to the apparatus or for securing sections of tissue that are removed from the membrane.

### TECHNICAL FIELD

The present invention relates to a medical device and method. More particularly, the present invention relates to an apparatus and method for creating an opening or orifice in a septum (or membrane). Specifically, the invention discloses an apparatus and methods for using the apparatus to make an opening in the membrane floor of a ventricle in the brain during an endoscopic third ventriculostomy (ETV) procedure.

### BACKGROUND OF THE INVENTION

Non-communicating hydrocephalus is a condition that results in the enlargement of the ventricles caused by abnormal accumulation of cerebrospinal fluid (CSF) within the cerebral ventricular system.

In non-communicating hydrocephalus there is an obstruction at some point in the ventricular system. The cause of non-communicating hydrocephalus usually is a congenital abnormality, such as stenosis of the aqueduct of Sylvius, congenital atresia of the foramina of the fourth ventricle, or spina bifida cystica. There are also acquired versions of hydrocephalus that are caused by a number of factors including subarachnoid or intraventricular hemorrhages, infections, inflammation, tumors, and cysts.

The main treatment for hydrocephalus is venticuloperitoneal (VP) shunts. The VP shunts are catheters that are surgically lowered through the skull and brain. The VP shunts are then positioned in the lateral ventricle. The distal end of the catheter is tunneled under the skin and positioned in the peritoneal cavity of the abdomen, where the CSF is absorbed.

However, the VP shunts have an extremely high failure rate, e.g., in the range of 30 to 40 percent. Failure includes clogging of the catheter, infection, and faulty pressure valves or one-way valves.

Another relatively newly re-introduced treatment for non-communicating hydrocephalus is the procedure known as an endoscopic third ventriculostomy (ETV). This procedure involves forming a burr hole in the skull. A probe is passed through the burr hole, through the cerebral cortex, through the underlying white matter and into the lateral and third ventricles. The probe is then used to create (fenestrate) an opening in the floor of the third ventricle and underlying membrane of Lillequist.

To verify that the procedure is successful, i.e., that an opening is formed in the floor of the third ventricle and the underlying membrane of Lillequist, the patient is observed with magnetic resonance imaging (MRI) after the puncture. The MRI is used to verify a flow of CSF through the opening in the floor of the third ventricle.

If the MRI is unable to detect the flow of CSF, a determination is made that an opening in the floor of the third ventricle was not formed, and the ETV procedure is repeated.

Since the MRI is typically located at a separate location, the ETV procedure typically requires the patient to be moved from location to location. This, in turn, increases the procedure time as well as the expense and complexity of the ETV procedure.

After the formation of an opening is verified, a catheter delivered balloon can be used to enlarge the opening. Even after successfully forming an opening in the floor of the third ventricle, the opening sometimes closes, typically within two weeks to two months after the ETV procedure. In this event, the patient will have to undergo another ETV procedure or risk serious injury or death. One potential reason for closing is that when the opening is fenestrated, it is formed as more of a rip such that the edges of the opening can appose and seal the opening closed.

Thus it would be beneficial to have a device and method for forming openings in the floor of the third ventricle that would allow a clinician to know that the opening had been formed without having to move the patient to a separate location for an MRI procedure. Such a device that would reduce the potential for the edges of openings to heal back together would be advantageous.

### SUMMARY OF THE INVENTION

It should be noted that the term distal end as used herein shall be taken to mean the end of the element being described that is furthest from a clinician who will be operating the apparatus. Stated another way, the distal end of an element is the first end of the element that will be inserted into the body of a patient when an apparatus of the type disclosed here is being used to remove a section of tissue from a tissue membrane in a body.

The current invention discloses preferred embodiments of an apparatus for creating an opening in a tissue membrane and methods for using the apparatus to create an opening in the floor of the third ventricle of a brain. Some embodiments of the apparatus comprise a cutting device that can include a cutting element and a resisting element located on the ends of elongate members. The cutting device can be manipulated so that the cutting element and resisting element are on opposite sides of a tissue membrane. Alternate preferred embodiments of the apparatus do not include a resisting element.

In some embodiments of the apparatus, the cutting device may be configured to tear a section of tissue from a tissue membrane rather than to cut the section away. However, all embodiments of the apparatus disclosed herein are intended to be used to create a hole in a tissue membrane by removing a portion of tissue from the membrane. This tissue may ultimately be removed from the body of a patient.

The cutting device is delivered to a tissue membrane using an elongate delivery device that can be routed through a working lumen in an endoscopic probe. The delivery device can also access the tissue membrane via means other than an endoscopic probe.

In one embodiment of a method for using the devices disclosed herein, a device is delivered to a location adjacent the floor of a third ventricle in a brain via and endoscopic probe through a burr hole in a skull. In another embodiment of a method for using the devices disclosed herein, the devices can be delivered to the floor of a third ventricle via a catheter that is navigated through the spinal subarachnoid space. The devices described herein can then be used to create an opening in the ventricle from the subarachnoid space side of the ventricular floor.

Embodiments of the invention can include a vacuum or suction lumen in the delivery device and a vacuum or suction source for stabilizing the tissue membrane relative to the apparatus and/or for removing sections of portions that are cut from the membrane (throughout this document, the term “vacuum” should be taken to mean “vacuum or suction”). The vacuum source can be any mechanical, electrical, or manually operated source that provides sufficient force for tissue stabilization relative to the cutting element (i.e., sufficient to force the tissue against a cutting edge of the device) and/or sufficient force to secure a portion of the tissue membrane for withdrawal from a body.

The present invention discloses methods and devices for creating an opening in the floor of a ventricle for performing an endoscopic third ventriculostomy. The foregoing and other features and advantages of the invention will become further apparent from the following detailed description of the presently preferred embodiments, read in conjunction with the accompanying drawings, which are not to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 is a cross-section view of a human cranium during an endoscopic third ventriculostomy (ETV) procedure using an endoscopic third ventriculostomy probe according to the present invention.

FIG. 2 is a perspective view of a cut-away section of an apparatus for creating an opening in a tissue membrane according to the present invention.

FIG. 3 is a cross sectional view of an apparatus for creating an opening in a tissue membrane according to the present invention.

FIGS. 4 through 8 are enlarged partial views of an apparatus according to the present invention being used to create an opening in the floor of a third ventricle during an ETV procedure.

FIG. 9 is a partial view of an alternate preferred embodiment of an apparatus according to the present invention.

FIG. 10 is an enlarged partial cross sectional view of the apparatus of the present invention shown in FIG. 9.

FIG. 11 is a block diagram representation of an apparatus according to the present invention.

### DETAILED DESCRIPTION

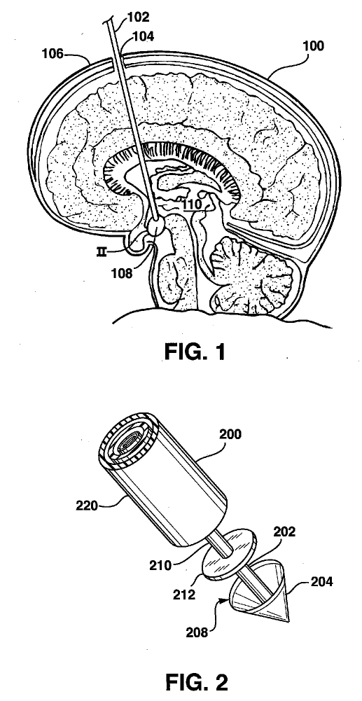

The invention will now be described in detail below by reference to the drawings, wherein like numbers refer to like structures. Referring to FIG. 1, one embodiment of an ETV procedure, using the devices and methods disclosed herein, includes forming a burr hole 104 (FIG. 1) in a skull 106, passing an endoscopic third ventriculostomy (ETV) device 102 through burr hole 104, and cutting an opening in the floor 108 of the third ventricle 110 with a cutting device (FIG. 2) to form an opening in floor 108. The ETV procedure can further include deploying a membrane eyelet into the opening.

The procedure can also include measuring the flow of CSF through the opening with a flow sensor, or using a pressure sensor to measure the pressure gradient across the opening. While not depicted in the drawings, the devices described herein can also include a lumen for injecting a contrast medium into the third ventricle such that a clinician can use fluoroscopy or other imaging modalities to determine if there is flow of CSF. The contrast injection lumen can be a separate lumen in the delivery devices described herein, or the contrast medium can be injected through a vacuum lumen or other lumen in the apparatus.

More particularly, FIG. 1 is a cross-section view of a human cranium 100 during an endoscopic third ventriculostomy (ETV) procedure using an endoscopic third ventriculostomy probe 102 according to the present invention. Initially, a burr hole 104 is formed in the skull 106. Probe 102 is passed through burr hole 104, through the cerebral cortex and through the underlying white matter to a location adjacent the floor 108 of the third ventricle 110 as illustrated in FIG. 1. An apparatus of the current invention is delivered through a lumen in the probe, and then used to create an opening in the floor 108 of third ventricle 110 and to remove a section of the ventricle floor and the underlying membrane of Lillequist.

It should be noted that for the purposes of this document, discussions and descriptions of creating openings in the floor of the third ventricle or removing sections of the ventricle floor are meant to include creating openings in or removing sections of the membrane of Lillequist. Thus any such discussion referring to the floor of a third ventricle should be read to include the underlying membrane of Lillequist regardless of whether the membrane is specifically referenced in the discussion or description.

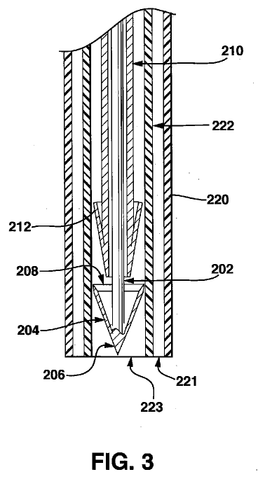

Referring to FIG. 2 and FIG. 3, there is shown one embodiment of an apparatus for removing a section of tissue from a tissue membrane according to the disclosure herein. The apparatus 200 includes a cutting device having a first elongate member 202 and a cutting element 204 disposed on the distal end of the first elongate member 202. The first elongate member is positioned in the interior of a generally tubular second elongate member 210 that has a resisting element 212 disposed on its distal end so that the first and second elongate members are coaxially aligned.

The cutting device is delivered to the third ventricle inside of an elongated delivery device 220. The delivery device depicted in FIGS. 2 & 3 includes a vacuum lumen 221, which can be connected to a vacuum source. The cutting device is disposed in a delivery lumen 223 that is defined by an interior member 222 and is oriented coaxially with the vacuum lumen 221 for a portion of the length of the delivery device. In other embodiments of the delivery device having vacuum lumens, the vacuum lumen is not coaxially oriented with other device members or lumens. In some embodiments of the apparatus, the interior member is an integral part of the delivery device 220. The interior member in at least one embodiment is a delivery sheath inserted into a separate delivery lumen that does not extend to the distal end of the delivery device.

In some embodiments of the invention, the delivery device can be inserted into a working lumen of an endoscopic third ventriculostomy probe such as the one shown in FIG. 1. In other embodiments, the delivery device can be inserted directly into a burr hole in a skull to perform a third ventriculostomy. In yet other embodiments of the invention, the elongated delivery device is a catheter. In at least one embodiment of the invention, the elongate delivery device is flexible. As noted above, and described in more detail below, the device can also be delivered to a location adjacent the floor of the third ventricle of the brain via a catheter that is navigated through the subarachnoid space.

The first elongate member 202 and the second elongate member 210 each have a distal end (as defined above) and a proximal end that may extend from a proximal end of the delivery device, so that a clinician will be able to use the elongate members 202 & 210 to manipulate the cutting element 204 and the resisting element 212. The elongate members can be constructed from the same material as the cutting element 204 and resisting element 212 or they can be constructed from other suitable biocompatible material that will allow a clinician sufficient control over the elements disposed on the distal end of the elongate members. In at least one embodiment of the apparatus, the elongate elements are flexible.

The cutting element 204 is disposed on the distal end of the first elongate member 202. The cutting element can be made from a biocompatible material, which may have shape memory properties. Examples of suitable materials include, but are not limited to, nitinol, stainless steel, a cobalt-based alloy, and MP35N®. In the embodiment depicted, the cutting element has a delivery configuration (as seen in FIG. 3) in which the element is collapsed such that it can be easily delivered through the delivery lumen 223 to a location in a body where the cutting device will be used. The cutting element also has a delivery configuration (as seen in FIG. 2) in which the element assumes a shape that will allow it to cut a section of tissue from a tissue membrane. In its deployed configuration, the diameter of the cutting element may be greater than the diameter of the delivery system.

As can be further seen in FIG. 3, the folded cutting element can be beveled or sharpened so that when the cutting element 204 of the depicted embodiment of the invention is in a collapsed delivery configuration, it forms a pointed end 206 on the distal most end of the first elongate member 202. When the element is delivered to a tissue membrane, the pointed end allows the cutting element to penetrate the membrane and assume the deployed configuration. When the cutting element is in a deployed configuration, a sharpened edge portion 208 on the base of the element is directed at the membrane. The shape of the cutting element allows a section of the tissue membrane to be removed such that an opening will remain in the membrane and CSF can flow through the opening.

The resisting element 212 is disposed on the distal end of the second elongate member 210. The resisting element can be made from a biocompatible material, which may have shape memory properties. Examples of suitable materials include, but are not limited to, nitinol, stainless steel, a cobalt-based alloy, and MP35N®. In the embodiment depicted, the resisting element has a delivery configuration (as seen in FIG. 3) in which the element is collapsed such that it can be easily delivered through the delivery lumen 223 to a location in a body where the resisting device will be used. The resisting element also has a delivery configuration (as seen in FIG. 2) in which the element assumes a generally planar configuration. In one preferred embodiment, the surface of the deployed resisting element is larger than the diameter of the base of the deployed cutting element. Another preferred embodiment has a resisting element with a surface that is smaller than the diameter of the base of the deployed cutting element, while yet another embodiment has a resisting element that is the same size as the diameter of the base of the deployed cutting element.

When the resisting element of the depicted embodiment is delivered to a tissue membrane, it will be deployed on the side of the membrane nearest to the delivery device while the cutting element is deployed on the opposite side of the membrane. The resisting element provides support to the tissue membrane and resistance to the sharpened edge portion on the base of the cutting element so that the cutting element can cut a section of tissue from the membrane. As will be explained below, when a clinician is using the embodiment of the invention depicted in FIGS. 2 & 3, the cutting element and the resisting element can be manipulated by pushing and pulling on the elongate members.

While the depicted embodiment shows the second elongate member having a channel communicating therethrough and the first elongate member disposed in that channel, in other embodiments of the invention the first elongate member is hollow and the second elongate member is disposed in the first elongate member. Additionally, at least one embodiment of the current invention does not include a resisting element.

In at least one embodiment, the position of the resisting element (supported by the second elongate member) and the cutting element (supported by the first elongate member) relative to the distal end of the device is reversed such that the resisting element pierces the tissue membrane. When the resisting element of this embodiment is in its delivery configuration, the end can be sharpened or beveled so that it forms a pointed end on the distal most end of the second elongate member. The cutting element of this embodiment is disposed on the end of the first elongate member such that the sharpened edge portion is oriented distally. In such an embodiment, the resisting element is inserted through the tissue membrane and the cutting element is deployed on the side of the tissue member nearest the deployment device.

The cutting element of the depicted device is conical in shape, but other embodiments of the cutting element have different shapes. At least one embodiment of the current invention has a parabolic/dome shaped cutting element and another embodiment has a ring shaped cutting element that is attached to the elongate member with a plurality of attachment members. In another embodiment, the cutting element is wire that is shaped such that it can be manipulated to cut a section of tissue from a tissue membrane.

Embodiments of the cutting element and the resisting element may be made from a biocompatible material that has sufficient elastic properties to permit deformation from a deployment configuration into a delivery configuration and subsequent reformation of the members back into the deployment configuration. Other embodiments of the cutting elements and resisting elements can be deformed from a delivery configuration into a deployment configuration and then undergo subsequent reformation of the members back into the delivery or retrieval configuration.

Materials for use in making the various embodiments of the cutting element and resisting element include any biocompatible material. These materials may have shape memory properties. Such materials can include shape memory metals, shape memory alloys, and plastics having shape memory properties. Suitable materials also have properties that will allow a cutting edge to be sharp enough to cut through a tissue membrane. The cutting elements and resisting elements of the current invention can be attached to the elongate members using any suitable technique that is selected based on the materials used for the various components. Examples of suitable techniques include welding and soldering. Examples of suitable materials include, but are not limited to, nitinol, stainless steel, a cobalt-based alloy, and MP35N®.

In at least one embodiment of the cutting devices disclosed herein, the cutting element and the resisting element are formed in the deployment configuration. The formed members can be heat set to provide the shape memory so that the members can be placed in a delivery configuration, but will re-form into the deployment configuration after delivery to a tissue membrane. In other embodiments having one of a cutting element or a resisting element, that member is formed in the deployment configuration and can then be heat set so that it will re-form to the deployment configuration from the delivery configuration.

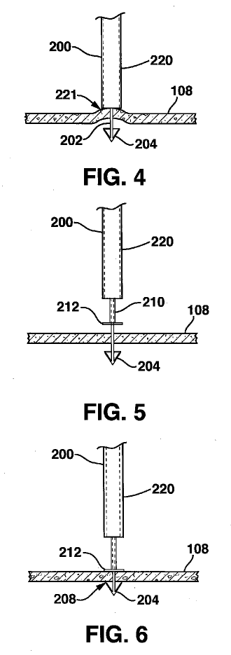

Referring now to FIGS. 4-8, there can be seen illustrations of the embodiment a cutting apparatus as disclosed herein and shown in FIGS. 2 & 3 being used to create an opening in the floor of a third ventricle and remove a section of tissue from the floor. After a ventriculostomy probe has been inserted in a burr hole in a cranium, the apparatus of the current invention is delivered to the ventricle. A safe area for cutting an opening in the ventricle floor is identified by endoscopic visualization, Doppler ultrasound, or other suitable visualization methods, such that the apparatus will not damage any arteries, other major blood vessels, the pituitary gland, or other significant structure.

Referring to FIG. 4, the delivery device 220 is then positioned directly adjacent to the identified safe area of the ventricle floor 108. The apparatus 200 can be delivered to the area adjacent the ventricle floor through a working lumen in the ventriculostomy probe, or separately from the probe. An exterior vacuum source can then be activated such that a sufficient vacuum is created in the vacuum lumen 221 to secure the ventricle floor 108 snugly to the distal end of the delivery device 220.

Once the ventricle floor is secured to the distal end of the delivery device, the cutting element 204 is deployed from the device and advanced through the ventricle floor. A clinician may advance the cutting element by pushing on the first elongate member 202. The vacuum source may then be deactivated or it can remain active through the remainder of the procedure. In various embodiments of a method for using an apparatus as disclosed herein, the vacuum source may be alternately activated and deactivated at various steps depending on the step being performed.

Referring now to FIG. 5 after the cutting element is advanced through the floor of the ventricle; the resisting element 212 is deployed by advancing it from the delivery device. A clinician may advance the resisting element by pushing on the second elongate member 210.

Referring to FIG. 6, after the cutting element 204 and resisting element 212 have been deployed, they are manipulated so that the sharpened edge portion 208 of the cutting element is directly against the ventricle floor on the opposite side of the floor from the resisting element. The resisting element is positioned to be directly against the floor of the ventricle opposite the cutting element. The resisting element will then provide support to the floor of the ventricle so that the sharpened edge portion 208 of the cutting element can make a clean cut.

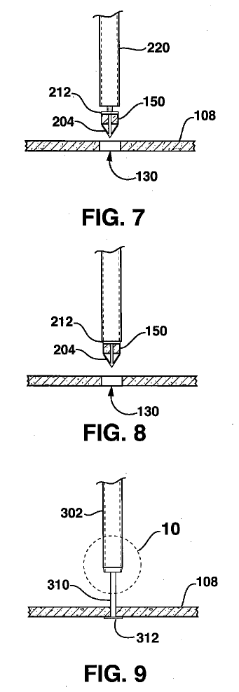

Referring to FIGS. 7 & 8, a clinician can then create an opening 130 in the ventricle floor by manipulating the first elongate member to pull the cutting element toward the resisting element while simultaneously manipulating the second elongate member so that the resisting element is urged toward the cutting element or at least held in one location. The resulting section of tissue 150 that is removed from the floor of the ventricle is secured inside the cutting element or in between the cutting element and the resisting element. The cutting element and the resisting element are then withdrawn to the distal end of the delivery device, which can then be withdrawn from the patient's body.

Once the tissue section has been removed, a clinician can evaluate the flow of CSF through the opening and place a flow meter in the body to monitor the flow if desired. A membrane eyelet can also be placed in the opening to further insure that the opening remains open.

In one embodiment of the apparatus of the current invention, the cutting device (cutting and resisting elements) is withdrawn completely into the delivery device after a section of tissue has been removed from the floor of the ventricle. In another embodiment, the vacuum source may be activated to assist in securing tissue that is removed from the ventricle floor so that it can be withdrawn from a patient's body. In another embodiment of the invention, the tissue section that is removed from a tissue membrane is not withdrawn from a patient's body.

The size of the opening in the floor of the third ventricle will vary based on the age of the patient, the preference of the clinician, and other factors. Preferred embodiments of the devices disclosed herein have cutting elements for creating openings that range in size from 1 mm to 15 mm in diameter. One preferred embodiment of the invention has a cutting element for creating an opening smaller than 1 mm and at least one other embodiment has a cutting element for creating an opening larger than 15 mm. Embodiments of the devices disclosed herein can have cutting elements and resisting elements that are larger in diameter than a delivery device after they are deployed from the delivery device. In at least one embodiment, the diameter of the opening created in the ventricle floor will be larger than the diameter of the delivery device.

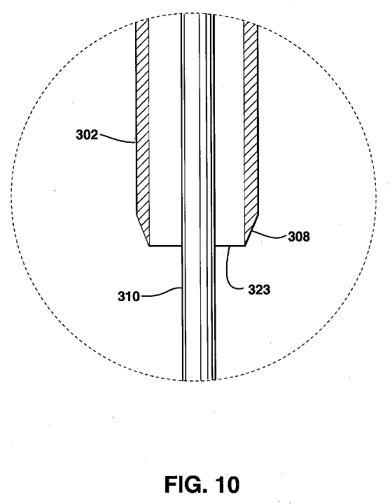

FIG. 9 and FIG. 10 illustrate another embodiment of an apparatus of the current invention. The cutting device is comprised of a first elongate member 302 having a sharpened edge portion 308 on the distal end of the member, a second elongate member 310, and a resisting element 312 disposed on the end of the second elongate member. The first elongate member 302 has at least a delivery lumen 323 extending therethrough so that the second elongate member 310 can be disposed inside of the first elongate member.

As described above, the resisting element is made from a biocompatible material having shape memory properties. The resisting element has a delivery configuration in which the element is collapsed such that it can be easily delivered through the delivery lumen 323 to a location in a body where the resisting device will be used. When the resisting element is in the delivery configuration, the folded element can be sharpened or beveled so that it forms a point on the distal most end of the second elongate member, that can easily penetrate the floor of a third ventricle. The resisting element also has a deployment configuration in which the element assumes a generally planar configuration that is larger in diameter than the diameter of the sharpened edge portion of the cutting element. Another preferred embodiment has a resisting element with a surface that is smaller than the diameter of the base of the cutting element, while yet another embodiment has a resisting element that is the same size as the diameter of the base of the cutting element.

The first elongate member can also serve as the delivery device for the apparatus shown in FIGS. 9 & 10. The apparatus can include a vacuum source. In some embodiments of the apparatus disclosed herein, a vacuum lumen may be in communication with the delivery lumen such that the vacuum lumen does not extend to the distal most tip of the delivery device.

To use the embodiment of apparatus disclosed in FIG. 9, a ventriculostomy probe is first inserted in a burr hole in a cranium, the apparatus of the current invention is delivered to the ventricle. A safe area for cutting an opening in the ventricle floor is identified by endoscopic visualization, Doppler ultrasound, or other suitable visualization methods, such that the apparatus will not damage any arteries, other major blood vessels, the pituitary gland, or other significant structure. The apparatus is delivered through a working lumen in the ventriculostomy probe, or separately from the probe to a position adjacent to the tissue of the safe area on the floor of the ventricle.

An exterior vacuum source can then be activated to secure the ventricle floor 108 snugly against the distal most end of the first elongate member 302. The resisting element 312 is then deployed from the first elongate member such that it is on the opposite side of the floor from the sharpened edge portion of the first elongate member.

The sharpened edge portion (308) of the first elongate member and the resisting element (312) are then manipulated so that they are directly against the floor of the ventricle on opposite sides of the floor. A clinician can then create an opening in the ventricle floor by manipulating the first elongate member to push the cutting element toward the resisting element while simultaneously manipulating the second elongate member so that the resisting element is urged toward the cutting element or at least moving one of the elements while the other is held in one location. The resulting section of tissue that is removed from the floor of the ventricle is secured inside the cutting element. The cutting element and the resisting element are then withdrawn to the distal end of the delivery device, which can then be withdrawn from the patient's body. In at least one embodiment of the invention, a vacuum source is activated to assist in securing tissue that is cut from the ventricle floor so that it can be withdrawn from a patient's body. In another embodiment, tissue that is removed from a tissue membrane is not removed from a patient's body.

Once the tissue section has been removed, a clinician can evaluate the flow of CSF through the opening and place a flow meter in the body to monitor the flow if desired. The clinician can also use a pressure sensor to monitor the pressure gradient of the CSF across the opening. A membrane eyelet can also be placed in the opening to further insure that the opening remains open.

The size of the opening in the floor of the third ventricle will vary based on the age of the patient, the preference of the clinician, and other factors. Embodiments of the apparatus described herein have cutting elements for creating openings in the range of sizes from 1 mm to 15 mm in diameter. One preferred embodiment of the invention has a cutting element for creating an opening smaller than 1 mm and at least one other embodiment has a cutting element for creating an opening larger than 15 mm. In at least one embodiment, the diameter of the opening created in the ventricle floor will be larger than the diameter of the delivery device.

One embodiment of an apparatus of the current invention has a cutting element similar to the cutting element shown in FIGS. 9 & 10, but does not include a resisting element. When using that embodiment, a vacuum source is used to secure the ventricle floor snugly to the distal end of the first elongate element. The first elongate element can then be rotated to cut through the floor of the ventricle. Alternately, the sharpened edge portion may be honed such that the vacuum source will provide sufficient force to create an opening in the floor of the ventricle.

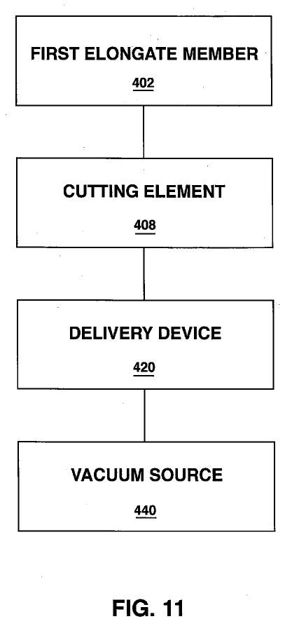

Referring to FIG. 11, there is shown a schematic representation of an embodiment of an apparatus for creating an opening in the floor of a third ventricle according to the current invention. The depicted embodiment includes a first elongate member 402, a cutting element 408, a delivery device 420, and a vacuum source 440. Other embodiments of the current invention can include a second elongate member, and a resisting element. Still other embodiments of the invention are configured to remove a portion of tissue from a tissue membrane by tearing the tissue away from the membrane.

The apparatus of the current invention can also be delivered to a location adjacent to the floor of the third ventricle of a brain via a catheter inserted into the subarachnoid space. To accomplish such a delivery, the clinician must first determine that subarachnoid access (for example by cervical, thoracic, or lumbar puncture) could be performed without risk of cerebral herniation. The clinician would then perform, for example, a lumbar puncture with an introducer and attach a Touhy Borst valve. A catheter would then be inserted through the valve and introducer. The catheter would be navigated superiorly within the spinal subarachnoid space to a location adjacent the floor of the third ventricle. The procedure described herein would then be performed from the subarachnoid space side of the third ventricle floor.

This disclosure provides exemplary embodiments of an apparatus that can be used for creating an opening in a tissue membrane and methods of using the apparatus to create an opening in the floor of a third ventricle. The scope of the present invention is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. An apparatus for creating an opening in a tissue membrane in a body comprising:
a cutting device having at least a first elongate member and a cutting element;
the first elongate member having a distal end and a proximal end;
the cutting element being disposed on the distal end of the first elongate member, the cutting element comprising at least an edge portion that is shaped to remove a section of tissue from the tissue membrane;
means to deliver the cutting device to a location adjacent to a tissue membrane in a body and
means to withdraw a tissue section from a body after the tissue section has been removed from a tissue membrane.

2. The apparatus of claim 1 wherein the cutting element is made from material having shape memory properties.

3. The apparatus of claim 1 wherein the cutting element has a delivery configuration so that it can be delivered to a location adjacent to a tissue membrane in a body and a deployment configuration; and
when the cutting element is in the deployment configuration, at a location adjacent to a tissue membrane in a body, the edge portion is oriented toward the tissue membrane.

4. The apparatus of claim 1 wherein the cutting element has a shape selected from the group of shapes consisting of a dome and a cone;
the edge portion is located at the base of the cutting element; and
the cutting element is disposed on the distal end of the first elongate member such that when the cutting element is in a deployment configuration, at a location adjacent to a tissue membrane in a body, the base of the cutting element is oriented toward the tissue membrane.

5. The apparatus of claim 1 wherein when the cutting element is in a delivery configuration it creates a generally pointed end on the distal end of the first elongate member, such that the pointed end created by the cutting element can pierce a tissue membrane in a body.

6. The apparatus of claim 1 wherein the cutting device further comprises a second elongate member and a resisting element;
the second elongate member having a distal end and a proximal end;
the resisting element being disposed on the distal end of the second elongate member; and
the resisting element being generally planar in shape;
whereby the resisting element has a size and configuration such that the resisting element can provide resistive stabilization to a tissue membrane in a body so that the cutting element will remove a section of tissue from the tissue membrane.

7. The apparatus of claim 6 wherein the resisting element is made from material having shape memory properties.

8. The apparatus of claim 6 wherein the resisting element has a delivery configuration so that it can be delivered to a location adjacent to a tissue membrane in a body and a deployment configuration, and
when the resisting element is in the deployment configuration the sharpened edge portion of the cutting element is oriented toward the resisting element.

9. The apparatus of claim 6 wherein when the resisting element is in a delivery configuration it creates a generally pointed end on the distal end of the second elongate member, such that the pointed end created by the resisting element can pierce a tissue membrane in a body.

10. The apparatus of claim 6 wherein one of the first elongate member or the second elongate member is a hollow tubular member with a channel communicating through the length of the member such that it has an interior and an exterior and the other of the first elongate member or the second elongate member is positioned in the interior of the hollow elongate member.

11. The apparatus of claim 6 wherein when the cutting device is deployed adjacent a tissue membrane in a body, the cutting element and the resisting element are on opposite sides of the tissue membrane and when the device is used to remove a section of tissue from the tissue membrane, the section of tissue is held between the cutting element and the resisting element for withdrawal from the body.

12. The apparatus of claim 1 wherein the means to deliver the cutting device to a location adjacent to a tissue membrane in a body is an essentially hollow, elongate delivery device having a delivery lumen communicating lengthwise therethrough.

13. The apparatus of claim 12 wherein the delivery device further comprises a vacuum lumen and a vacuum source.

14. The apparatus of claim 13 wherein the delivery lumen and the vacuum lumen are co-axially aligned at the distal end of the delivery device.

15. The apparatus of claim 12 wherein the delivery device further comprises a hollow delivery sheath.

16. The apparatus of claim 13 wherein when the cutting device is used to remove a section of tissue from a tissue membrane in a body, the vacuum source is activated to secure the section of tissue to the distal end of the delivery device for removal from the body.

17. The apparatus of claim 1 wherein the tissue membrane is the floor of the third ventricle of a brain and the apparatus further comprises a means for introducing a contrast medium into the third ventricle.

18. An apparatus for creating an opening in the floor of a third ventricle of a brain comprising:
a cutting device having at least a first elongate member and a cutting element;
the first elongate member having a distal end and a proximal end;
the cutting element being disposed on the distal end of the first elongate member, the cutting element comprising at least an edge portion that is shaped to remove a section of tissue from the tissue membrane; and
means to deliver the cutting device to a location adjacent to a tissue membrane in a body.

19. An apparatus for creating an opening in the floor of a third ventricle of a brain comprising:
a cutting device having at least a first elongate member, a second elongate member, a cutting element, and a resisting element;
the first elongate member and the second elongate member each having a distal end and a proximal end;
the cutting element and the resisting element each being made from material having shape memory properties and each having a delivery configuration, so that each can be delivered to a location adjacent to a floor of a third ventricle in a body;
the resisting element being disposed on the distal end of the second elongate member and the resisting element further having a generally planar deployment configuration with a size and configuration such that the resisting element can provide resistive stabilization to a floor of a third ventricle in a body so that the cutting element will cut a section of tissue from the floor of a third ventricle;
the cutting element having a shape selected from the group of shapes consisting of a dome and a cone with a sharpened edge portion located at the base of the cutting element;
the cutting element being disposed on the distal end of the first elongate member such that when the cutting element is in a deployment configuration, the base of the cutting element is oriented toward the resisting element;
the sharpened edge portion being shaped to cut a section of tissue from a floor of a third ventricle in a body;
means to deliver the cutting device to a location adjacent to a floor of a third ventricle in a body; and
means to withdraw a tissue section from a body after the tissue section has been cut out of a floor of a third ventricle.

20. The apparatus of claim 19 wherein when the cutting element is in a delivery configuration it creates a generally pointed end on the distal end of the first elongate member, such that the pointed end created by the cutting element can pierce a floor of a third ventricle in a body.

21. The apparatus of claim 19 wherein when the resisting element is in a delivery configuration it creates a generally pointed end on the distal end of the second elongate member, such that the pointed end created by the resisting element can pierce a floor of a third ventricle in a body.

22. The apparatus of claim 19 wherein one of the first elongate member or the second elongate member is a hollow tubular member with a channel communicating through the length of the member such that it has an interior and an exterior and the other of the first elongate member or the second elongate member is positioned in the interior of the hollow elongate member such that the first elongate member and the second elongate member are co-axially aligned.

23. The apparatus of claim 19 wherein when the cutting device is deployed adjacent a floor of a third ventricle in a body, the cutting element and the resisting element are on opposite sides of the floor of the third ventricle and when the device is used to cut a section of tissue from the floor of a third ventricle, the section of tissue is held between the cutting element and the resisting element for removal from the body.

24. The apparatus of claim 19 wherein the means to deliver the cutting device to a location adjacent to a floor of a third ventricle in a body is an essentially hollow, elongate delivery device having a delivery lumen communicating lengthwise therethrough;
the device further having a vacuum lumen communicating at least partially therethrough and
the apparatus further comprises a vacuum source.

25. The apparatus of claim 24 wherein the delivery device may further comprise a hollow delivery sheath.

26. An apparatus for creating an opening in a tissue membrane in a body comprising:
a cutting device having at least a first elongate member and a cutting element;
the first elongate member having a distal end, a proximal end, and a channel communicating lengthwise therethrough such that the first elongate member is essentially tubular;
the cutting element being formed by a sharpened beveled edge at the distal end of the first elongate, essentially tubular member;
the cutting element being shaped to completely cut a section of tissue from the tissue membrane;
the apparatus further comprising an essentially hollow, elongate delivery device to deliver the cutting device to a location adjacent to a tissue membrane in a body, the delivery device having a delivery lumen communicating lengthwise therethrough; and
means to withdraw a tissue section from a body after the tissue section has been cut out of a tissue membrane.

27. The apparatus of claim 26 wherein the cutting device further comprises a second elongate member, having a distal end and a proximal end, and a resisting element;
the second elongate member being disposed in the channel of the first elongate member and;
the resisting element being disposed on the distal end of the second elongate member; and
the resisting element being generally planar in shape;
whereby the resisting element has a size and configuration such that the resisting element can provide resistive stabilization to a tissue membrane in a body so that the cutting element will cut a section of tissue from the tissue membrane.

28. The apparatus of claim 27 wherein the resisting element is made from material having shape memory properties and the resisting element has a delivery configuration so that it can be delivered to a location adjacent to a tissue membrane in a body and a deployment configuration; and
when the resisting element is in the deployment configuration the sharpened edge portion of the cutting element is oriented toward the resisting element.

29. The apparatus of claim 28 wherein when the resisting element is in a delivery configuration it creates a generally pointed end on the distal end of the second elongate member, such that the pointed end created by the resisting element can pierce a tissue membrane in a body.

30. The apparatus of claim 26 wherein the delivery device further comprises a vacuum lumen and the apparatus further comprises a vacuum source.

31. The apparatus of claim 26 wherein the delivery device further comprises a hollow delivery sheath.

32. The apparatus of claim 26 wherein the tissue membrane is the floor of the third ventricle of a brain and the apparatus further comprises means for, introducing a contrast medium into the third ventricle.

33. A method for creating an opening in the third ventricle of a brain comprising the steps of:
providing an apparatus for creating an opening in the floor of the third ventricle, the apparatus comprising at least a cutting device, a means for delivering the device to the third ventricle, and means for removing tissue portions from a body;
accessing the floor of the third ventricle of a brain;
deploying the cutting device;
manipulating the cutting device to cut a portion of tissue from the floor of the third ventricle;
securing the tissue portion; and withdrawing the tissue portion and the cutting device from the third ventricle.
